(19) United States
(12) Patent Application Publication
(10) Pub. No.: US 2017/0035911 A1
(43) Pub. Date: Feb. 9, 2017

(54) Amorphous Cobicistat Solid Dispersion

(71) Applicant: MYLAN LABORATORIES LTD., Jubilee Hills, Hyderabad (IN)

(72) Inventors: Ramakoteswara Rao JETTI, Hyderabad (IN); Balakrishna Reddy BHOGALA, Hyderabad (IN); Aggi Rami Reddy BOMMAREDDY, Hyderabad (IN); Anjaneya Raju INDUKURI, Hyderabad (IN)

(21) Appl. No.: 15/039,774

(22) PCT Filed: Nov. 28, 2014
(86) PCT No.: PCT/IB2014/066414

§ 371 Date: May 26, 2016

(30) Foreign Application Priority Data
Nov. 29, 2013 (IN) 5508/CHE/2013

(51) Int. Cl. A61K 47/48; A61K 31/5377; A61K 9/10

(52) U.S. Cl. CPC A61K 47/48969; A61K 9/10; A61K 31/5377

(57) ABSTRACT

The present disclosure relates to a solid dispersion of amorphous cobicistat and methods of its preparation. Cobicistat may be complexed with a pharmaceutically acceptable carrier such as β-cyclodextrin or hydroxy! propyl β-cyclodextrin. The present disclosure also provides pharmaceutical formulations for administration to patients and methods of their use.

### CROSS-REFERENCE TO RELATED APPLICATIONS

This application claims the benefit of earlier Indian provisional patent application No. 5508/CHE/2013, filed on Nov. 29, 2013.

### BACKGROUND OF THE INVENTION

Field of the Disclosure

The present disclosure is directed to an amorphous solid dispersion comprising cobicistat, and one or more pharmaceutically acceptable carrier, a pharmaceutical composition comprising the amorphous solid dispersion as well as a process for obtaining the same.

Background of the Disclosure

Cobicistat is chemically known as 1,3-thiazol-5-ylmethyl [(2R,5R)-5-{[(2S)2-[(methyl {[2-(propan-2-yl)-1,3-thiazol-4-yl]methyl}carbamoyl)amino]-4-(morpholin-4-yl)butanoyl]amino}-1,6-diphenylhexan-2-yl]carbamate (Formula I).

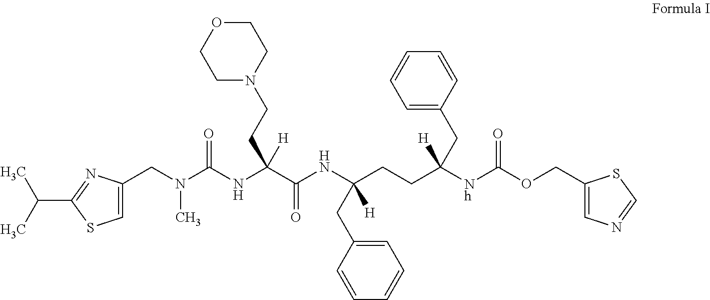

International patent publication WO2008010921 describes compounds and pharmaceutical compositions to improve the pharmacokinetics of a co-administered drug by inhibiting cytochrome P450 monooxygenase.

International patent publication WO2009135179 (which is hereby incorporated by reference) discusses the difficulties associated with processing the compound of formula (I), the solid state properties of the compound of formula (I) make it difficult to handle and process on a large scale. For example, its low glass transition temperature, hygroscopicity, and lack of crystallinity, as well as its non-free flowing nature make it particularly difficult to process and to formulate (e.g., as a tablet).

The present invention overcomes these limitations of the prior art through formulation of active pharmaceutical ingredients, such as cobicistat, through solid dispersion using a cyclodextrin carrier.

### SUMMARY OF THE DISCLOSURE

One aspect of the present disclosure is to provide a solid dispersion of amorphous cobicistat. In some embodiments, the cobicistat is complexed with a pharmaceutically acceptable carrier. The pharmaceutically acceptable carrier may be α-cyclodextrin, β-cyclodextrin, or γ-cyclodextrin. In some embodiments, the pharmaceutically acceptable carrier is hydroxypropyl-β-cyclodextrin. The solid dispersions of amorphous cobicistat-hydroxypropyl-β-cyclodextrin complexes have a powdered X-ray diffraction (PXRD) pattern as shown in FIGS. 1 and 2. The amorphous solid dispersions of the present invention may have a molar ratio of cobicistat and pharmaceutically acceptable carrier from about 1:0.75 to about 1:3. The amorphous solid dispersions of the present invention may have a weight ratio of cobicistat and pharmaceutically acceptable carrier from about 35:65 to about 90:10. The present invention also encompasses oral dosage forms that include these solid dispersions of amorphous cobicistat complexed with a pharmaceutically acceptable carrier.

Another aspect of the present disclosure is to provide a process for preparing solid dispersion of amorphous cobicistat comprising dissolving cobicistat in a solvent, contacting the solution with pharmaceutically accepted carrier capable of complexing cobicistat, followed by removing the solvent to isolate a solid dispersion of amorphous cobicistat. The solvent may be alcohol solvent, ketone solvent, chlorinated solvent, water, or mixtures thereof. In some embodiments, the alcohol solvent may be methanol, ethanol, propanol, isopropanol, n-butanol, sec-butanol, 2-butanol, t-butanol, pentanol, or mixtures thereof. In some embodiments, the ketone solvent may be acetone, methylethyl ketone, methylisobutyl ketone, 2-butanone, or mixtures thereof. The chlorinated solvent may be dichloromethane, dichloroethane, chloroform, carbon tetrachloride, or mixtures thereof.

The process of removing the solvent may be achieved by evaporation, distillation, spray drying, filtration, lyophillization, or agitated thin film drier (ATFD).

### BRIEF DESCRIPTION OF THE FIGURES

FIG. 1 is a representative powder x-ray diffraction (PXRD) pattern of amorphous cobicistat solid dispersion with hydroxy propyl β-cyclodextrin.

FIG. 2 is a representative PXRD pattern of amorphous cobicistat dispersion with β-cyclodextrin

### DETAILED DESCRIPTION OF THE DISCLOSURE

It is to be understood that the description of the present invention has been simplified to illustrate elements that are relevant for a clear understanding of the invention, while eliminating, for purposes of clarity, other elements that may be well known.

The present disclosure relates to an amorphous solid dispersion of cobicistat and process for the preparation thereof. The present disclosure also relates to a process for formulating different pharmaceutical compositions of amorphous cobicistat.

One embodiment of the present disclosure provides a solid dispersion of amorphous cobicistat.

Within the context of the present invention, a solid dispersion may be a molecular dispersion of a compound, particularly a drug substance within a carrier matrix. Formation of a molecular dispersion provides a means of reducing the particle size, in some embodiments, to nearly molecular levels. As the carrier dissolves, the drug is exposed to the dissolution media as fine particles that are typically amorphous. The fine particles dissolve and absorb more rapidly than larger particles.

The term “solid dispersion” as used herein, refers to a system in a solid state including at least two components, wherein one component is dispersed throughout the other component or components. The term “amorphous solid dispersion” as used herein, refers to stable solid dispersions comprising amorphous drug substance and a carrier matrix. An “amorphous drug substance” as used herein, is an amorphous solid dispersion containing drug substance in a substantially amorphous solid state form. A substantially amorphous state may include at least about 80%, at least about 90%, or at least 95% of the drug substance in the dispersion is in an amorphous form.

Another embodiment of the present disclosure is to provide a process for the preparation of amorphous cobicistat complex with a pharmaceutically acceptable carrier according to the steps:
a) dissolving cobicistat in a solvent to form a solution,
b) contacting the solution with one or more pharmaceutically acceptable carrier capable of complexing cobicistat;
c) removing the solvent, and
d) isolating the solid dispersion of amorphous cobicistat.

According to the present embodiment, cobicistat may initially be dissolved in a solvent. The obtained solution may then be treated with a pharmaceutically acceptable carrier capable of complexing cobicistat. The resultant clear solution may then be stirred for 25-30 minutes. Solvent may be removed using standard laboratory techniques as discussed more fully below to obtain amorphous cobicistat solid dispersion.

As used herein, the term “solvent,” unless otherwise indicated, refers to an alcohol solvent, ketone solvent, chlorinated solvent, water, or a mixture thereof.

As used herein, alcohol solvents include, but are not limited to, methanol, ethanol, propanol, isopropanol, n-butanol, sec-butanol, 2-butanol, t-butanol, pentanol, or mixtures thereof; ketone solvents include, but are not limited to, acetone, methylethyl ketone, methylisobutyl ketone, 2-butanone or mixtures thereof and chlorinated solvents include, but are not limited to, dichloromethane, dichloroethane, chloroform, carbon tetrachloride, or mixtures thereof.

According to the present embodiment, the solvent may be removed by known techniques which may include, but are not limited to, evaporation, distillation, spray drying, filtration, lyophillization, or agitated thin film drier (ATFD).

According to the present disclosure, the starting material cobicistat may be crystalline, amorphous, or semi-solid in nature.

Another embodiment of the present disclosure provides a pharmaceutical composition including amorphous cobicistat and at least one pharmaceutically acceptable carrier. In certain embodiments, the pharmaceutically acceptable carrier may be α-, β-, or γ-cyclodextrin. In certain embodiments, it has been found that hydroxypropyl-β-cyclodextrin and β-cyclodextrin are particularly useful.

In some embodiments of the present invention, the molar ratio of an amorphous solid dispersion of cobicistat and hydroxypropyl-β-cyclodextrin complex is about 1:0.75 to about 1:3. In certain embodiment, the molar ratio is about 1:0.75, 1:1.5, 1:2 or 1:3.

In some embodiments of the present invention, the wt/wt ratios of an amorphous solid dispersion of cobicistat to a hydroxypropyl-β-cyclodextrin or β-cyclodextrin complex is about 35:65 to about 90:10.

According to the present disclosure, a reduced amount of cyclodextrin may be used to obtain a solid dispersion when compared to prior art formulations that utilize cobicistat silicon dioxide. Within the context of the present invention, cobicistat-cyclodextrin complex may be employed at ranges down to about 5% wt/wt of the formulation.

Yet another embodiment of the present disclosure provides a pharmaceutical composition which may minimize gastrointestinal side effects and promote internal absorptions to increase a bioavailability by enhancing the solubilization of water-insoluble drugs, and a method of manufacturing the same.

In accordance with one embodiment of the present disclosure, the dissolution properties of drugs may be improved by their conversion to an amorphous state or by complexation with cyclodextrins. The present disclosure provides a pharmaceutical composition including a solid dispersion which may be prepared by dissolving a water-insoluble drug and a substituted cyclodextrin in an organic solvent with or without water to make a mixture. The mixture may then be dried under a reduced pressure or spray dried.

According to the present disclosure, the preparation of cobicistat complexed with α-cyclodextrin, β-cyclodextrin, γ-cyclodextrin, with some embodiments employing hydroxypropyl-β-cyclodextrin, may be achieved in the following manner. Initially cobicistat and the cyclodextrin may be dissolved in a solvent. The solvent may then be removed, yielding solids which could be directly compressed to tablets that dissolve completely within minutes when ingested by patients. Amorphous water soluble derivatives of cyclodextrins are potent, nontoxic solubilizers of drugs and lipids.

Yet another embodiment of the present disclosure provides an amorphous solid dispersion of cobicistat in combination with pharmaceutically acceptable carriers, which may be formulated into tablets, capsules, suspensions, dispersions, injectables, or other pharmaceutical forms.

In some embodiments, the amorphous solid dispersions of cobicistat of the present invention may be included in tablets for oral administration. One of skill in the art will recognize a wide variety of pharmaceutically acceptable excipients that may be included in such a tablet formulation, including lactose monohydrate, microcrystalline cellulose, croscarmelose sodium, hydroxypropyl cellulose, sodium lauryl sulfate, and magnesium stearate.

The cobicistat dispersions disclosed herein may be combined with additional active pharmaceutical components for the treatment of viral infections, including HIV protease inhibitors, HIV non-nucleoside inhibitors of reverse transcriptase, HIV nucleoside inhibitors of reverse transcriptase, HIV nucleotide inhibitors of reverse transcriptase, HIV integrase inhibitors, ap41 inhibitors, CXCR4 inhibitors, gp120 inhibitors, G6PD inhibitors, NADH-oxidase inhibitors, CCR5 inhibitors, capsid polymerization inhibitors, other drugs for treating HIV, interferons, ribavirin, NS3 protease inhibitors, alpha-glucosidase 1 inhibitors, hepatoprotectants, non-nucleoside inhibitors of HCV, NS5a inhibitors, NS5b polymerase inhibitors, other drugs for treating HCV, and combinations thereof. In some particularly useful embodiments, the amorphous solid dispersions of cobicistat disclosed herein may be formulated in a solid dosage form that may include elvitegravir, emtricitabine, tenofovir disoproxil fumarate, atazanavir, or combinations thereof,

Solid State Stability Data:

The solid state stability of the amorphous cobicistat solid dispersions in different ratios of cobicistat to β-cyclodextrin and hydroxypropyl cyclodextrin as prepared in example 6-13 were determined by storing the sample at long term (25±2° C. & 60±5% RH) conditions and 2-8° C. for 15 days, 1 month, 2 months, 3 months and 6 months. The samples are tested by PXRD and HPLC for final purity and degradation products. The results are given in the following table.

| Condition | PXRD | Purity (%) |
|---|---|---|
| Initial | Amorphous | 99.8 |
| After 15 days | Amorphous | 99.7 |
| After 1 month | Amorphous | 99.7 |
| After 2 month | Amorphous | 99.7 |
| After 3 month | Amorphous | 99.7 |
| After 6 month | Amorphous | 99.6 |

The different ratios of amorphous cobicistat solid dispersions are found to be chemically and physically very stable. The PXRD pattern remains same as the initial PXRD pattern and there is no degradation observed by HPLC.

The following examples are provided for illustrative purposes only and are not intended to limit the scope of the invention in anyway.

### EXAMPLES

Powder X-Ray Diffraction (PXRD)

The PXRD patterns of said polymorphs of the invention were measured on a Bruker D8 Discover powder diffractometer equipped with a goniometer of q/q configuration and LynxEye detector. The Cu-anode X-ray tube was operated at 40 kV and 30 mA. Experiments were conducted over the 20 range of about 2.0°-50.0°, a step size of about 0.030°, and about a 50 seconds step time.

### Example 1

### Preparation of Amorphous Cobicistat Solid Dispersion (1:1 Mole Ratio)

Cobicistat silicon dioxide (2 gm, 1:1) was suspended in dichloromethane (40 mL) and stirred for 4-5 minutes. The suspension was filtered and washed with dichloromethane (10 mL). The filtrate was concentrated using a rotary-evaporator under reduced pressure at 25° C. to provide cobicistat as a gummy mass (0.75 gm, 0.966 mmoles). The gummy mass was dissolved in methanol (5 mL) at 25° C. and hydroxypropyl-β-cyclodextrin (1.3 gm, 1.16 mmoles) in methanol (10 mL) was added. The mixture was stirred for 30 minutes. The resultant clear solution was concentrated on a rotary-evaporator under reduced pressure at 40° C. to provide cobicistat as an amorphous foamy solid (1.7 gm).

### Example 2

### Preparation of Amorphous Cobicistat Solid Dispersion (1:2 Mole Ratio)

Cobicistat silicon dioxide (1 gm, 1:1) was suspended in dichloromethane (15 mL) and stirred for 30 minutes. The suspension was filtered and washed with dichloromethane (5 mL). The filtrate was concentrated using a rotary-evaporator under reduced pressure to provide cobicistat as a gummy mass (0.45 gm, 0.58 mmoles). Hydroxypropyl-β-cyclodextrin (1.6 gm, 1.16 mmoles) and methanol (20 mL) were added to the gummy mass. The mixture was stirred for 30 minutes. The resultant clear solution was concentrated on a rotary-evaporator under reduced pressure at 40° C. to provide cobicistat as an amorphous foamy solid (1.8 gm).

### Example 3

### Preparation of Amorphous Cobicistat Solid Dispersion (1:3 Mole Ratio)

Cobicistat silicon dioxide (1 gm, 1:1) was suspended in dichloromethane (20 mL) and stirred for 30 minutes. The suspension was filtered and washed with dichloromethane (5 mL). The filtrate was concentrated using a rotary-evaporator under reduced pressure to provide cobicistat as a gummy mass (0.45 gm, 0.58 mmoles). Hydroxypropyl-β-cyclodextrin (2.4 gm, 1.74 mmoles) and methanol (20 mL) were added to the gummy mass. The mixture was stirred for 30 minutes. The resultant clear solution was concentrated on a rotary-evaporator under reduced pressure at 40° C. to provide cobicistat as an amorphous foamy solid (2.8 gm).

### Example 4

### Preparation of Amorphous Cobicistat Solid Dispersion (1:0.75 Mole Ratio)

Cobicistat silicon dioxide (2 gm, 1:1) was suspended in dichloromethane (40 mL) and stirred for 30 minutes. The suspension was filtered and washed with dichloromethane (10 mL). The filtrate was concentrated using a rotary-evaporator under reduced pressure to provide cobicistat as a gummy mass (0.9 gm, 1.16 mmoles). Hydroxypropyl-β-cyclodextrin (1.2 gm, 0.87 mmoles) and methanol (20 mL) were added to the gummy mass and stirred for 30 min. The resulting clear solution was concentrated on a rotary-evaporator under reduced pressure at 40° C. to provide cobicistat as an amorphous foamy solid (2.3 gm),

### Example 5

### Preparation of Amorphous Cobicistat Solid Dispersion (1:1.5 Mole Ratio)

Cobicistat silicon dioxide (1 gm, 1:1) was suspended in dichloromethane (20 mL) and stirred for 30 minutes. The suspension was filtered and washed with dichloromethane (5 mL). The filtrate was concentrated using a rotary-evaporator under reduced pressure to provide cobicistat as a gummy mass (0.4 gm, 0.515 mmoles). Hydroxypropyl-β-cyclodextrin (1.1 gm, 0.773 mmoles) and methanol (20 mL) were added to the gummy mass and stirred for 30 min. The resultant clear solution was concentrated on a rotary-evaporator under reduced pressure at 40° C. to provide cobicistat as an amorphous foamy solid (1.5 gm).

### Example 6

### Preparation of Amorphous Cobicistat Solid Dispersion (90:10 wt/wt Ratio)

Cobicistat (10 gm) was dissolved in methanol (100 mL) at 25-30° C., hydroxypropyl-β-cyclodextrin (1.1 gm) was added at 25-30° C., and the mixture was stirred for 10-15 minutes to get a clear solution. The resulting clear solution was filtered through hyflo to remove any undissolved particulate and dried to yield amorphous cobicistat in a laboratory Spray Dryer (Model Buchi-290) with the feed rate of the solution at 10 ml/min and inlet temperature at 45° C.

### Example 7

### Preparation of Amorphous Cobicistat Solid Dispersion (75:25 wt/wt Ratio)

Cobicistat (10 gm) was dissolved in methanol (130 mL) at 25-30° C. and hydroxypropyl-β-cyclodextrin (3.3 gm) was added at 25-30° C. and stirred for 10-15 minutes to get clear solution. The resulting clear solution was filtered through hyflo to remove any undissolved particulate and dried to yield amorphous solid dispersion of cobicistat in a laboratory Spray Dryer (Model Buchi-290) with feed rate of the solution 10 ml/min and inlet temperature at 45° C.

### Example 8

### Preparation of Amorphous Cobicistat Solid Dispersion (50:50 wt/wt Ratio)

Cobicistat (8 gm) was dissolved in methanol (120 mL) at 25-30° C., hydroxypropyl-β-cyclodextrin (8 gm) was added at 25-30° C., and the mixture was stirred for 10-15 minutes to get clear solution. The resulting clear solution was filtered through hyflo to remove any undissolved particulate and dried to yield amorphous cobicistat in a laboratory Spray Dryer (Model Buchi-290) with feed rate of the solution 10 ml/min and inlet temperature at 45° C.

### Example 9

### Preparation of Amorphous Cobicistat Solid Dispersion (35:65 wt/wt Ratio)

Cobicistat (8 gm) was dissolved in methanol (120 mL) at and hydroxypropyl-β-cyclodextrin 14.9 was added. The solution was stirred at 25-30° C. for 10-15 minutes to get clear solution. The resulting clear solution was filtered through hyflo to remove any undissolved particulate and dried to yield amorphous cobicistat in a laboratory spray dryer (Model Buchi-290) with feed rate of the solution 10 ml/min and inlet temperature at 45° C.

### Example 10

### Preparation of Amorphous Cobicistat Solid Dispersion (90:10 w/w Ratio)

β-cyclodextrin (1 gm) was dissolved in water (60 mL) at 25-30° C. To the clear solution, cobicistat (9 gm) and methanol (120 mL) were added and stirred for 10-15 minutes to get a clear solution. The resulting clear solution was filtered through hyflo. The bed was then washed with a mixture of methanol (60 mL) and water (30 mL) to remove any undissolved particulate, then dried in a laboratory spray dryer (Model Buchi-290) with feed rate of the solution 10 ml/min and inlet temperature at 50° C. to yield amorphous cobicistat.

### Example 11

### Preparation of Amorphous Cobicistat Solid Dispersion (75:25 w/w Ratio)

β-cyclodextrin (3.33 gm) was dissolved in water (200 mL) at 25-30° C. To the clear solution, cobicistat (10 gm) and methanol (300 mL) were added, and the solution was stirred for 10-15 minutes to get a clear solution. The resulting clear solution was filtered through hyflo to remove any undissolved particulate, and dried in a laboratory spray dryer (Model Buchi-290) with feed rate of the solution 10 ml/min and inlet temperature at 50° C. to yield amorphous cobicistat.

### Example 12

### Preparation of Amorphous Cobicistat Solid Dispersion (50:50 w/w Ratio)

β-cyclodextrin (6 gm) was dissolved in water (420 mL) at 25-30° C. To the clear solution, cobicistat (6 gm) and methanol (420 mL) were added and the solution was stirred for 10-15 minutes to get a clear solution. The resulting clear solution was filtered through hyflo to remove any undissolved particulate, and the solution was subjected to spray drying in a laboratory spray dryer (Model Buchi-290) with feed rate of the solution 10 ml/min and inlet temperature at 60° C. to yield amorphous cobicistat.

### Example 13

### Preparation of Amorphous Cobicistat Solid Dispersion (35:65 w/w Ratio)

β-cyclodextrin (9.75 gm) was dissolved in water (630 mL) at 25-30° C. To the clear solution, cobicistat (6.5 gm) and methanol (400 mL) were added and the solution was stirred for 10-15 minutes to get a clear solution. The resulting clear solution was filtered through hyflo to remove any undissolved particulate, and the solution was subjected to spray drying in a laboratory spray dryer (Model Buchi-290) with feed rate of the solution 10 ml/min and inlet temperature at 60° C. to yield amorphous cobicistat.

We claim:

1. An amorphous solid dispersion of cobicistat, comprising cobicistat complexed with a pharmaceutically acceptable carrier.

2. The amorphous solid dispersion of claim 1, wherein the pharmaceutically acceptable carrier is α-cyclodextrin, β-cyclodextrin, or γ-cyclodextrin.

3. The amorphous solid dispersion of claim 1, wherein the pharmaceutically acceptable carrier is hydroxypropyl-β-cyclodextrin.

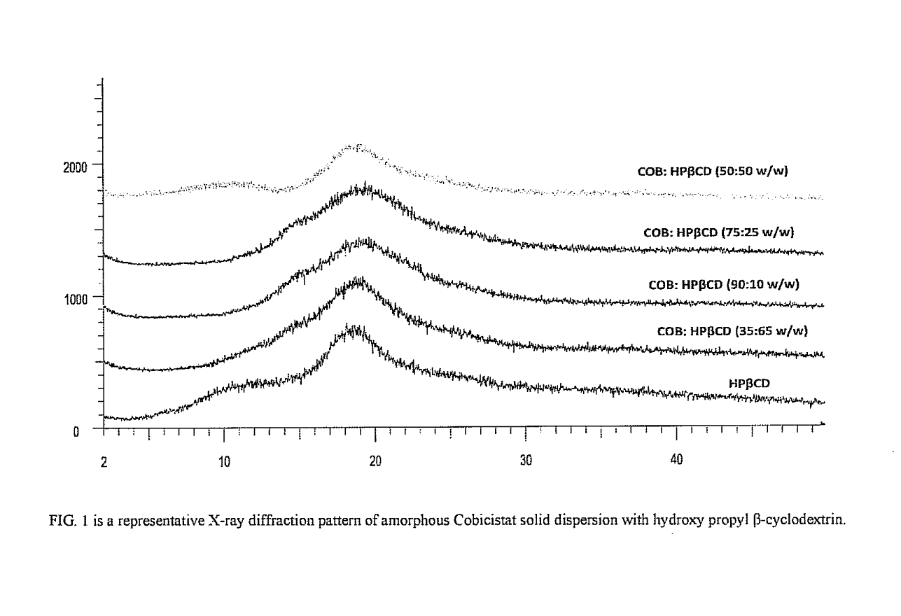

4. The amorphous of solid dispersion of claim 3, having a powdered X-ray diffraction pattern as shown in FIG. 1.

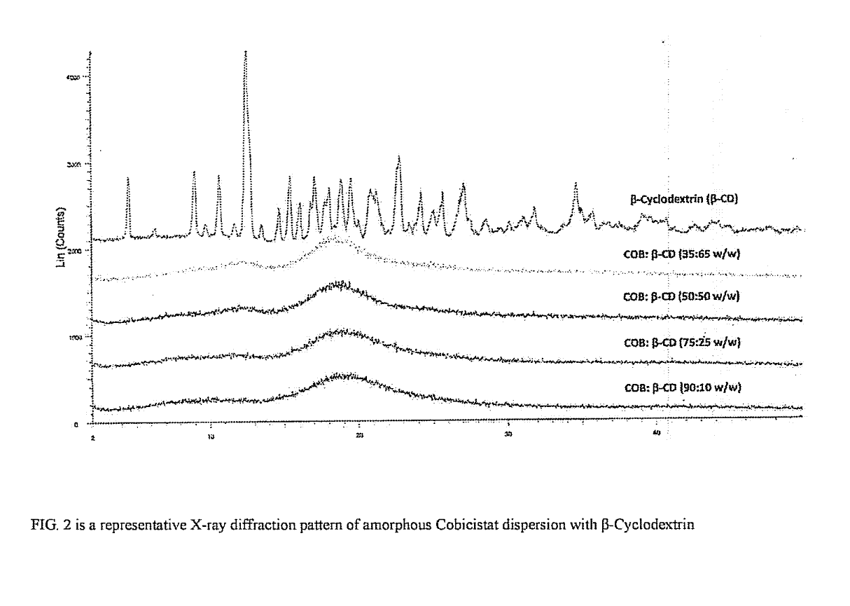

5. The amorphous of solid dispersion of claim 2, having a powdered X-ray diffraction pattern as shown in FIG. 2.

6. The amorphous solid dispersion of claim 1, containing said cobicistat and said pharmaceutically acceptable carrier in a molar ratio from about 1:0.75 to about 1:3.

7. The amorphous solid dispersion of claim 1, containing said cobicistat and said pharmaceutically acceptable carrier are present in a wt/wt ratio from about 35:65 to about 90:10.

8. A process for preparation of a solid dispersion of amorphous cobicistat comprising the steps of:
a) dissolving cobicistat solvent to form a solution;
b) contacting the solution with a pharmaceutically acceptable carrier capable of complexing cobicistat;
removing the solvent; and
d) isolating the solid dispersion of amorphous cobicistat.

9. The process according to claim 8, wherein the solvent is selected from the group consisting of alcohol solvent, ketone solvent, chlorinated solvent, water, and mixtures thereof.

10. The process according to claim 9, wherein the alcohol solvent is selected from the group consisting of methanol, ethanol, propanol, isopropanol, n-butanol, sec-butanol, 2-butanol, t-butanol, pentanol, and mixtures thereof.

11. The process according to claim 9, wherein the ketone solvent is selected from the group consisting of acetone, methylethyl ketone, methylisobutyl ketone, 2-butanone, and mixtures thereof.

12. The process according to claim 9, wherein said chlorinated solvent is selected from the group consisting of dichloromethane, dichloroethane, chloroform, carbon tetrachloride, and mixtures thereof.

13. The process according to claim 8, wherein the pharmaceutically acceptable carrier is α-cyclodextrin, β-cyclodextrin, or γ-cyclodextrin.

14. The process according to claim 8, where the pharmaceutically acceptable carrier is hydroxypropyl-β-cyclodextrin or β-cyclodextrin.

15. The process according to claim 8, wherein said removing step is achieved by evaporation, distillation, spray drying, filtration, lyophillization, or agitated thin film drier (ATFD).

16. The process according to claim 8, containing said cobicistat and said pharmaceutically acceptable carrier in a molar ratio from about 1:0.75 to about 1:3.

17. The process according to claim 8, containing said cobicistat and said pharmaceutically acceptable carrier in a wt/wt ratio from about 35:65 to about 90:10.

18. A oral pharmaceutical dosage form, comprising the amorphous solid dispersion of cobicistat as recited in one of claims 1-7.
